(19) United States
(12) Patent Application Publication
(10) Pub. No.: US 2013/0317052 A1
(43) Pub. Date: Nov. 28, 2013

(54) QUINOLINE DERIVATIVE COMPOUND, METHOD FOR PREPARING SAME, AND PHARMACEUTICAL COMPOSITION CONTAINING SAME

(71) Applicants: Miwon Son, Gyeonggi-do (KR); Tae Ho Lee, Gyeonggi-do (KR); Sang Zin Choi, Gyeonggi-do (KR); Nam Joon Baek, Seoul (KR); Kang Ro Lee, Gyeonggi-do (KR); Ki Hyun Kim, Gyeonggi-do (KR); Soon Hoe Kim, Gyeonggi-do (KR)

(72) Inventors: Miwon Son, Gyeonggi-do (KR); Tae Ho Lee, Gyeonggi-do (KR); Sang Zin Choi, Gyeonggi-do (KR); Nam Joon Baek, Seoul (KR); Kang Ro Lee, Gyeonggi-do (KR); Ki Hyun Kim, Gyeonggi-do (KR); Soon Hoe Kim, Gyeonggi-do (KR)

(73) Assignee: Dong-A ST Co., Ltd., Seoul (KR)

(21) Appl. No.: 13/989,048

(22) PCT Filed: Nov. 24, 2010
(86) PCT No.: PCT/KR2010/008345

§ 371 Date: Jul. 12, 2013

(51) Int. Cl. C07D 455/03

(52) U.S. Cl. CPC C07D 455/03
USPC 514/289; 546/73

(57) ABSTRACT

The present invention relates to a novel quinoline derivative compound, an optical isomer thereof, a pharmaceutically acceptable salt thereof, and a hydrate or a solvate thereof. The novel quinoline derivative compound, the optical isomer thereof, the pharmaceutically acceptable salt thereof, and the hydrate or the solvate thereof accelerates gastrointestinal movement, and thus can effectively prevent or treat gastrointestinal mobility disorders.

### TECHNICAL FIELD

The present invention relates to a quinoline derivative compound, an optical isomer thereof, a pharmaceutically acceptable salt thereof, or a hydrate or solvate thereof, a preparation method thereof, and a pharmaceutical composition comprising the same.

### BACKGROUND ART

Pathological mechanisms of functional gastrointestinal diseases such as gastrointestinal motility disorders act together, manifesting one or usually multiple symptoms. Depending on the symptoms, functional gastrointestinal diseases are classified into ulcer-like dyspepsia, dysmotility-like dyspepsia, reflux-like dyspepsia, nonspecific or unspecified functional dyspepsia, and the like.

These functional gastrointestinal diseases are observed to have a worldwide morbidity of 25 to 50%, and patients in need of treatment account for about 5% of all diagnosed cases. Incidence of functional gastrointestinal diseases exhibits a high regional fluctuation. For example, there is a relatively low pathogenic occurrence in the occident, e.g., 22% for the U. K. and 19% for the U.S, whereas the incidence increases to 35˜42% in Japan and to 62% in East Africa. Digestive tract diseases rank as the second most abundant among all diseases in South Korea. Particularly, the prevalence of gastric diseases is high, with functional gastrointestinal disorders diagnosed in 30˜40% of Korean population. Since functional gastrointestinal diseases exhibit high morbidity and severe symptoms, leading to a deterioration in the quality of a patient's life and a need for frequent medical examination and treatment, thus presenting inconvenience to daily work schedule, an effective treatment is necessary.

Symptomatic causes of these functional gastrointestinal diseases usually remain unclear, although some of them have been identified. Such cryptogenic functional gastrointestinal diseases cannot be defined by histopathological and biochemical organic lesions, and are therefore explained in terms of functional symptoms. With regard to cryptogenic functional gastrointestinal diseases, treatment is directed towards reducing symptoms. Various symptoms that manifest in functional gastrointestinal disorders may be treated mainly by promoting gastrointestinal motility.

Functional dyspepsia, which is a representative gastrointestinal motility disorder, is also diagnosed based on various dyspepsia symptoms without apparent organic lesions, and therefore therapeutic treatment thereof is not simple, and most symptoms fluctuate between amelioration and deterioration, depending on various factors including diet and stress. These pathological mechanisms act together, manifesting one or usually multiple symptoms. For example, symptoms of functional dyspepsia include post-meal satiety, anorexia, a sense of abdominal fullness, early satiety, belching, epigastric discomfort or pain, brash, nausea, vomiting, acid reflux, and heartburn, and all symptoms of functional dyspepsia still remain unknown in terms of pathophysiology (Panganamamula et. al., Functional (Nonulcer) Dyspepsia, Current Treatment Options in Gastroenterology, 5, pp. 153-160, 2002).

Representative among therapeutics for functional dyspepsia are domperidone, metoclopramide, levosulpiride, mosapride, itopride, and erythromycin, which are all gastrointestinal prokinetic agents. For the treatment of brash and ulcer, which are representative symptoms of functional dyspepsia, gastric acid suppressants and gastric antacids are used, but these drugs such as H2 antagonists, usually have temporary effects (Vincenzo Stanghellini et al., Delayed Gastric Emptying in Functional Dyspepsia, Current Treatment Options in Gastroenterology, 7, 259-264, 2004).

5-HT4 receptor agonists, a kind of gastroprokinetic agents which have recently been used as therapeutics for functional dyspepsia, were found to improve the symptoms without increasing a strain in the gastric fundus. Cisapride, one of these 5-HT4 receptor agonists, functions to promote gastric emptying, with a high statistic significance compared to other drugs. As to the duodenum or intragastric pressure wavelength (6>cm), however, cispride increases thresholds for the perception of gastric distension in healthy individuals as well as in patients, and may also exhibit adverse side effects on the heart that are potentially fatal.

Other conventional gastroprokinetic agents are far inferior to cisapride in therapeutic effect, and there is therefore a pressing need to develop a therapeutic for functional dyspepsia or gastrointestinal motility disorders that exerts excellent therapeutic effects, without provoking side effects.

### DISCLOSURE

### Technical Problem

The present invention provides a novel quinoline derivative compound, an optical isomer thereof, a pharmaceutically acceptable salt thereof, and a hydrate or solvate thereof.

The present invention provides a method for preparing a novel quinoline derivative compound, an optical isomer thereof, a pharmaceutically acceptable salt thereof, or a hydrate or solvate thereof.

The present invention provides a pharmaceutical composition for the prevention or treatment of a gastrointestinal motility disorder, comprising a novel quinoline derivative compound, an optical isomer thereof, a pharmaceutically acceptable salt thereof, or a hydrate or solvate thereof.

The present invention provides the use of a composition comprising a novel quinoline derivative compound, an optical isomer thereof, a pharmaceutically acceptable salt thereof, or a hydrate or solvate thereof in the prevention or treatment of a gastrointestinal motility disorder.

The present invention provides a method for preventing or treating a gastrointestinal motility disorder using a quinoline derivative compound, an optical isomer thereof, a pharmaceutically acceptable salt thereof, or a hydrate or solvate thereof.

### Technical Solution

The present invention provides a quinoline derivative compound represented by the following Chemical Formula 1, an optical isomer thereof, a pharmaceutically acceptable salt thereof, and a hydrate or solvate thereof:

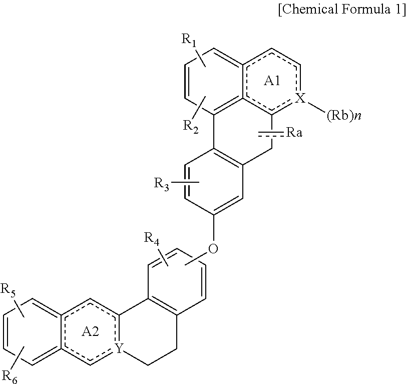

wherein for Chemical Formula 1:

R₁ to R₆ are independently —H; or methoxy,

Rₐ is —H; methoxy; or —O—,

[symbol] represents a single bond; or a double bond, with the proviso that when [symbol] represents the single bond, Ra is —H or methoxy; or when [symbol] represents the double bond, Ra is —O—,

X and Y are independently —N—; or —N⁺—,

Rb is —H; or linear or branched alkyl of C₁ to C₄,

n means the number of Rb bound with X and is an integer of 0 or 1;

A1 and A2 are independently a 6-membered cycloaliphatic ring; or a 6-membered aromatic ring, with the proviso that when A1 is the 6-membered cycloaliphatic ring, n is 1, or when A1 is the 6-membered aromatic ring, n is zero.

In Chemical Formula 1, X and Y may be the same or different. For example, both X and Y may be N. Alternatively, X may be N while Y is N+.

In Chemical Formula 1, A1 and A2 may be independently an aliphatic ring or an aromatic ring. For example, A1 may be the cycloaliphatic ring while A2 is the aromatic ring. Alternatively, A1 may be the aromatic ring while A2 is the cycloaliphatic ring.

When A1 or A2 is the cycloaliphatic ring, the quinoline derivative compound represented by Chemical Formula 1 may have an asymmetric carbon atom in the cycloaliphatic ring A1 or A2. In this context, the compound of Chemical Formula 1, an optical isomer, and a racemic mixture thereof may fall within the scope of the present invention.

In accordance with the present invention, the quinoline derivative compound represented by Chemical Formula 1 may be a compound represented by the following Chemical Formula 2 or 3:

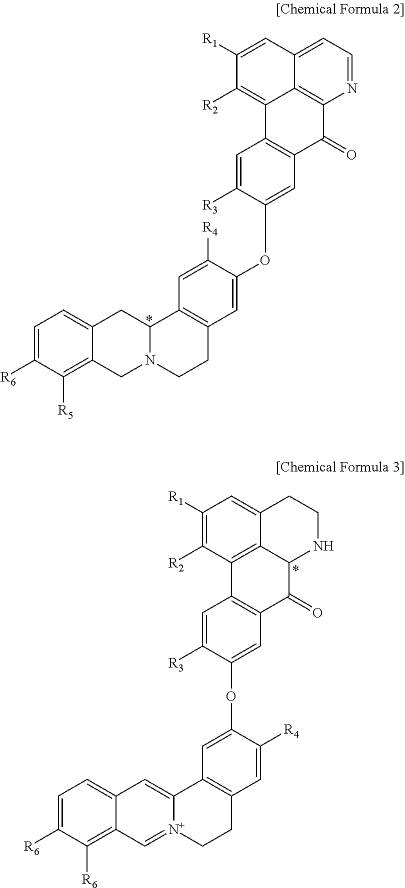

wherein for Chemical Formula 2 or 3:

R₁ to R₆ are independently —H; or methoxy, and

* represents an asymmetric carbon atom.

As can be seen in Chemical Formula 2 or 3, the compound may have an asymmetric carbon atom. Thus, the present invention envisages the compound represented by Chemical Formula 2 or 3, and an optical isomer thereof.

The present invention includes the following compounds, an optical isomer thereof, a pharmaceutically acceptable salt thereof, a hydrate thereof, and a solvate thereof.
1,2,10-Trimethoxy-9-(2,9,10-trimethoxy-5,8,13,13a-tetrahydro-6H-isoquino[3,2-a]isoquinolin-3-yloxy)-dibenzo[de,g]quinolin-7-one; or
3,9,10-Trimethoxy-2-(1,2,10-trimethoxy-5,6,6a,7-tetrahydro-4H-dibenzo[de,g]quinolin-9-yloxy)-5,6-dihydro-isoquino[3,2-a]isoquinolinylium

Preferably, the present invention includes the following compounds, an optical isomer thereof, a pharmaceutically acceptable salt thereof, a hydrate thereof, and a solvate thereof:
(S)-1,2,10-Trimethoxy-9-(2,9,10-trimethoxy-5,8,13,13a-tetrahydro-6H-isoquino[3,2-a]isoquinolin-3-yloxy)-dibenzo[de,g]quinolin-7-one; or
(S)-3,9,10-Trimethoxy-2-(1,2,10-trimethoxy-5,6,6a,7-tetrahydro-4H-dibenzo[de,g]quinolin-9-yloxy)-5,6-dihydro-isoquino[3,2-a]isoquinolinylium.

As used herein, the term “pharmaceutically acceptable salt” refers to any of typical salts employed in the pharmaceutical field. Examples of the salts include inorganic ion salts, such as salts of calcium, potassium, sodium and magnesium, inorganic acid salts, such as salts of hydrochloric acid, nitric acid, phosphoric acid, bromic acid, iodic acid, perchloric acid, tartaric acid, and sulfuric acid, organic acid salts, such as salts of acetic acid, trifluoroacetic acid, citric acid, maleic acid, succinic acid, oxalic acid, benzoic acid, tartaric acid, fumaric acid, mandelic acid, propionic acid, citric acid, lactic acid, glycolic acid, gluconic acid, galaturonic acid, glutamic acid, glutaric acid, glucuronic acid, aspartic acid, ascorbic acid, carbonic acid, vanillic acid, hydroiodic acid, mandelic acid, mucic acid, nitric acid, pamoic acid, pantothenic acid, succinic acid, and tartaric acid, and sulfonic acid salts, such as salts of methane sulfonic acid, ethane sulfonic acid, benzene sulfonic acid, p-toluene sulfonic acid, camphoric acid, and naphthalene sulfonic acid, amino acid salts such as salts of glycine, arginine, and lysine, and amine salts, such as salts of triethyl amine, triethylamine, ammonia, pyridine, and picoline, but are not limited thereto. For example, pharmaceutically acceptable salts are of hydrochloric acid as the inorganic acid salts and methane sulfonic acid as the organic acid salt, respectively, which may be used as the pharmaceutically acceptable salts of the present invention.

A hydrate of the compound of Chemical Formula 1, 2 or 3, the above-suggested compounds, optical isomers or the pharmaceutically acceptable salts thereof may include a stoichiometric or non-stoichiometric amount of water bound by non-covalent intermolecular forces. The hydrate may include at least one equivalent of water, for example, one to five equivalents of water. It may be prepared by crystallizing the compound of Chemical Formula 1, 2 or 3, an optical isomer thereof, or a pharmaceutically acceptable salt thereof in water or an aqueous solvent.

A solvate of the compounds represented by Chemical Formula 1, 2 or 3, the above-suggested compounds, or the optical isomers or the pharmaceutically acceptable salts thereof may comprise a stoichiometric or non-stoichiometric amount of a solvent bound by non-covalent intermolecular forces. Preferred is a solvent which is non-volatile, non-toxic, and suitable for administration to humans. Ethanol, methanol, propanol, and methylene chloride may be exemplary.

The compounds represented by Chemical Formula 1, 2, or 3, the above-suggested compounds, the optical isomers or the pharmaceutically acceptable salts thereof are prophylactic or therapeutic for gastrointestinal motility disorders. Having affinity for dopamine-D2 receptors, serotonin-1A receptors, and serotonin-1B receptors, the compounds of the present invention, that is, the compounds represented by Chemical Formula 1, 2, or 3, the above-suggested compounds, or the optical isomers or the pharmaceutically acceptable salts thereof can be used for the prevention or treatment of various symptoms of gastrointestinal motility disorders through interaction with the receptors.

The present invention provides a method for preparing a compound represented by the following Chemical Formula 1, an optical isomer thereof, a pharmaceutically acceptable salt thereof, or a hydrate or solvate thereof, comprising 1) reacting a compound represented by the following Chemical Formula 4 or an optimal isomer thereof with a sulfonyl compound in the presence of a base to synthesis a compound represented by the following Chemical Formula 5 or an optical isomer thereof; and 2) reacting the compound of Chemical Formula 5 or the optimal isomer thereof with a compound represented by the following Chemical Formula 6 in the presence of a base.

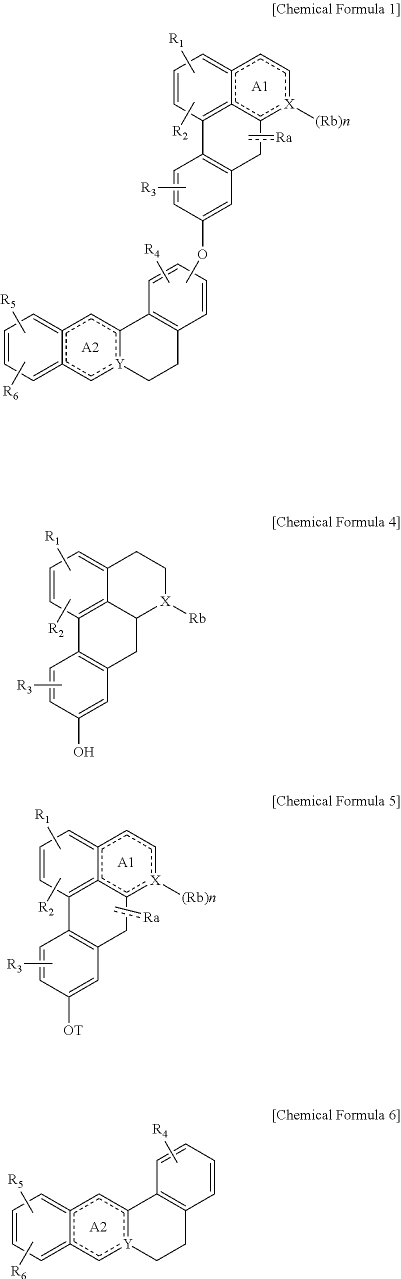

wherein for Chemical Formulas 1, 4, 5 and 6:

R₁ to R₆ are independently H or methoxy;

Ra is —H, methoxy or —O—;

[symbol] represents a single bond or a double bond, with the proviso that when [symbol] represents the single bond, Ra is —H or methoxy, or when [symbol] represents the double bond, Ra is —O—;

X and Y are independently —N— or —N⁺—;

Rb is —H or linear or branched alkyl of C₁ to C₄;

n means the number of Rb bound with X and is an integer of 0 or 1;

A1 and A2 are independently a 6-membered cycloaliphatic ring or a 6-membered aromatic ring, with the proviso that when A1 is the 6-membered cycloaliphatic ring, n is 1, or when A1 is the 6-membered aromatic ring, n is zero; and

T is linear or branched alkyl sulfonyl of C₁ to C₄, linear or branched fluoroalkyl sulfonyl of C₁ to C₄, or substituted or unsubstituted arylsulfonyl of C₃ to C₁₀.

In Chemical Formulas 1, 4, 5 and 6, X and y may be the same or different. For example, both X and Y may be N. Alternatively, X may be N while Y is N+.

The compound represented by Chemical Formula 4 or 6, or the optical isomer thereof may be prepared using a typical method known in the art, or may be commercially available. For example, the compound may be obtained from an extract of Corydalis tuber using a well-known method (Tae Ho Lee et al., Biol. Pharm. Bull. 33(6) 958-962 (2010), “Effects of Corydaline from Corydalis Tuber on Gastric Motor Function in an Animal Model”, Ki Hyun Kim et al., Planta Med. 2010 May 27, “New Cytotoxic Tetrahydroprotoberberine-Aporphine Dimeric and Aporphine Alkaloids from Corydalis Turtschaminovii”).

Each reaction step of the method of the present invention may be carried out in an organic solvent. No particular limitations are burdened on the kind of the organic solvent. For example, methanol, ethanol, propanol, dimethylsulfoxide (DMSO), tetrahydrofuran (THF), acetonitrile (ACN), dimethylformamide (DMF), N-methylpyrrolidinone (NMP), dimethylacetamide (DMA), or a mixture thereof may be used as a solvent for the reactions of the preparation method.

In the preparation method of the present invention, the compound of Chemical Formula 4 or the optical isomer thereof is reacted with the sulfonyl compound to prepare a compound represented by Chemical Formula 5. No particular limitations are imparted to the kind of the sulfonyl compound as long as it can effectively substitute for —H of the compound of Chemical Formula 4 or the optical isomer thereof, and can leave from the compound of Chemical Formula 5 upon the subsequent reaction between the compound of Chemical Formula 5, and the compound of Chemical Formula 6 or the optical isomer thereof. The sulfonyl compound available for the reaction may be a sulfonyl halide such as sulfonyl chloride, sulfonyl bromide, and sulfonyl iodide, or a sulfonyl imide. For examples, the sulfonyl compound may include methylsulfonyl halides, ethylsulfonyl halides, propylsulfonyl halides, toluenesulfonyl halides, trifluoromethylsulfonyl halides, trifluoromethylsulfonyl halides, trifluorobenzenesulfonyl halides, N-phenyl bistrifluoromethyl sulfonyl imide, and N-butyl bistrifluoromethyl sulfonyl.

In accordance with the preparation of the chemical formula 5, an oxidation step may be further included just after the reaction of the compound of Chemical Formula 4 or the optical isomer thereof with a sulfonyl compound when A1 is the aromatic ring, [symbol] represents the double bond, and Ra is —O— in Chemical Formula 5.

In accordance with the preparation of the chemical formula 5, the reaction with the compound of Chemical Formula 6 may be carried out immediately after the reaction between the compound of Chemical Formula 4 or the optical isomer thereof and the sulfonyl compound when A1 is the cyclo-aliphatic ring, [symbol] represents the single bond, and Ra is —H or methoxy.

In the presence of the base, the compound of Chemical Formula 5 or the optical isomer thereof is reacted with the compound of Chemical Formula 6 or the optical isomer thereof to prepare the compound of Chemical Formula 1. The sulfonyl group in the compound of Chemical Formula 5 or the optical isomer thereof is a good leaving group, so that it can be readily substituted by the compound of Chemical Formula 6 or the optical isomer thereof to afford the compound of Chemical Formula 1.

In accordance with the method of the present invention, so long as it can readily leave from the compound of Chemical Formula 5 or the optical isomer thereof in reaction with the compound of Chemical Formula 6 or the optical isomer thereof, any solfonyl group may be employed. Examples of the sulfonyl group available for the reaction may include methylsulfonyl, ethylsulfonyl, propylsulfonyl, toluenesulfonyl, trifluoromethyl sulfonyl, trifluoromethyl sulfonyl, and trifluorobenzene sulfonyl, with a preference for methylsulfonyl, p-toluenesulfonyl or trifluoromethylsulfonyl.

In accordance with the method of the present invention, various bases may be employed. For instance, the base may be triethylamine, N,N-diisopropylethylamine, N-methylorphyrin, N-methylpiperidine, 4-dimethylaminopyridine, N,N-dimethylaniline, 2,6-rutidine, tricalcium phosphate, potassium carbonate, pyridine or a mixture thereof. The base may be preferably triethylamine, or tricalcium phosphate.

In the case where the compound of Chemical Formula 1 is specified into the compound of Chemical Formula 2, the preparation method of the present invention may be carried out by reacting a compound represented by the following Chemical Formula 4-1 or an optical isomer thereof with a sulfonyl compound to synthesize a compound represented by the following Chemical Formula 5-1 or an optical isomer thereof; oxidizing the compound of Chemical Formula 5-1 or the optical isomer thereof to prepare a compound represented by the following Chemical Formula 5-2; and reacting the compound of Chemical Formula 5-2 with a compound represented by the Chemical Formula 6-1 or an optical isomer thereof.

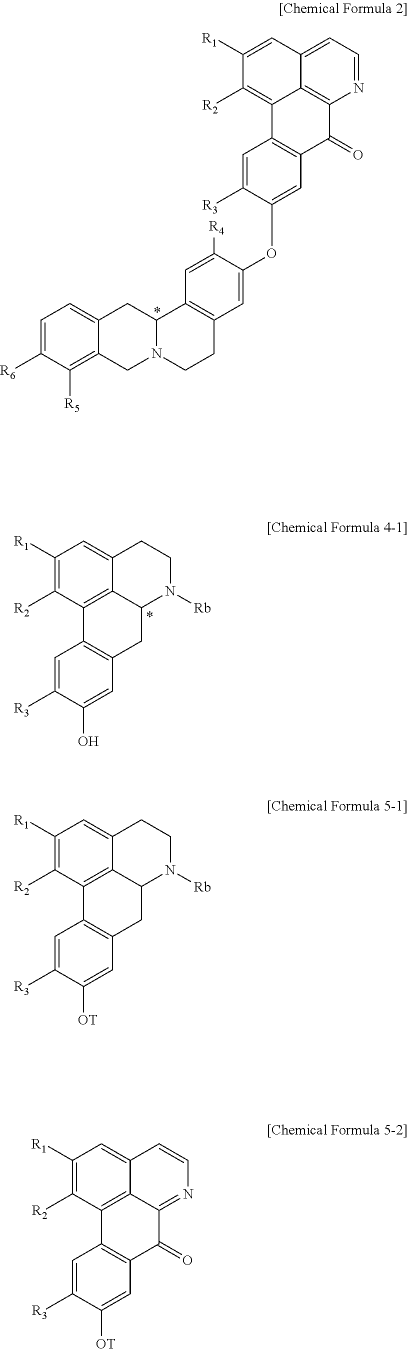

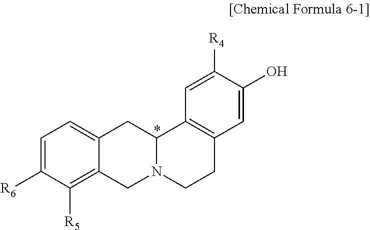

wherein for Chemical Formulas 2, 4-1, 5-1, 5-2 and 6-1:

R₁ to R₆ are independently —H or methoxy;

Rb is —H or a linear or branched alkyl of C₁ to C₄;

T is linear or branched alkyl sulfonyl of C₁ to C₄, linear or branched fluoroalkyl sulfonyl of C₁ to C₄, or substituted or unsubstituted arylsulfonyl of C₃ to C₁₀; and

* represents an asymmetric carbon atom.

When the compound of Chemical Formula 1 is specified into the compound of Chemical Formula 2, the preparation method of the present invention may be illustrated in the following Reaction Scheme 1:

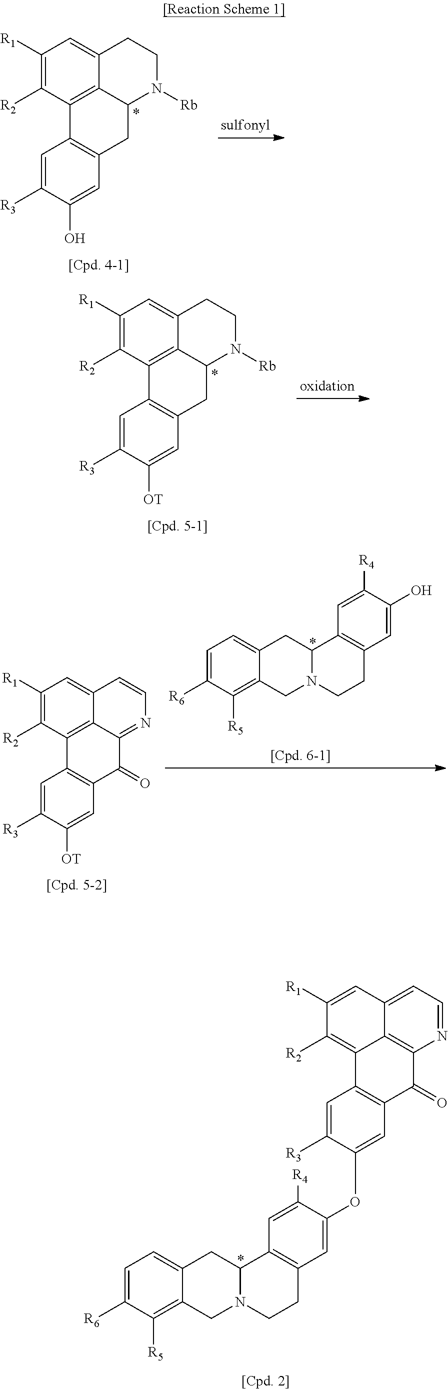

wherein for the reaction scheme 1:

R₁ to R₆ are independently —H or methoxy;

Rb is —H or linear or branched alkyl of C₁ to C₄;

T is linear or branched alkyl sulfonyl of C₁ to C₄, linear or branched fluoroalkyl sulfonyl of C₁ to C₄, or substituted or unsubstituted arylsulfonyl of C₃ to C₁₀; and

* represents an asymmetric carbon atom.

Preferably, in Reaction Scheme 1, R₁ to R₆ may be methoxy, Rb may be methyl, and T may be trifluoromethylsulfonyl.

In Reaction Scheme 1 according to the method of the present invention, compounds of Chemical Formulas 2, 4-1, 5-1 and 6-1 bear an asymmetric carbon atom at the position *. Therefore, in accordance with a method for preparing the compound represented by Chemical Formula 2, the optical isomer thereof, the pharmaceutically acceptable salt thereof, or the hydrate or solvate thereof, compounds represented by Chemical Formula 4-1, 5-1 and 6-1, or optical isomers thereof may be employed.

The compound of Chemical Formula 4-1 or 6-1, or the optical isomer thereof may be prepared using a typical method known in the art, or may be commercially available. For example, the compound may be obtained from an extract of Corydalis tuber using a well-known method (Tae Ho Lee et al., Biol. Pharm. Bull. 33(6) 958-962 (2010), “Effects of Corydaline from Corydalis Tuber on Gastric Motor Function in an Animal Model”, Ki Hyun Kim et al., Planta Med. 2010 May 27, “New Cytotoxic Tetrahydroprotoberberine-Aporphine Dimeric and Aporphine Alkaloids from Corydalis Turtschaminovii”).

As can be understood from Reaction Scheme 1 according to the method of the present invention, the optical structure of Chemical Formula 2 can be determined by that of the compound represented by Chemical Formula 6-1. Hence, when the compound of Chemical Formula 6-1 is a pure optical isomer, the compound of Chemical Formula 2 is obtained in the form of the predetermined optical structure. For example, if the compound of Chemical Formula 6-1 has an (S)-configuration, the compound of Chemical Formula 2 may be obtained as an (S) enantiomer.

On the other hand, when the compound of Chemical Formula 6-1 is a racemate, the compound of Chemical Formula 2 may be obtained in a racemic mixture. In this case, the racemic mixture can be separated into desired enantiomers of Chemical Formula 2 using a well-known method.

As illustrated in Reaction Scheme 1 according to the preparation method of the present invention, the compound of Chemical Formula 5-1 or the optical isomer thereof may be synthesized from the compound of Chemical Formula 4-1 or the optical isomer thereof by reaction with the sulfonyl compound in the presence of the base. No particular limitations are imported to the kind of the sulfonyl compound If it is reacted with the compound of Chemical Formula 4-1 or the optical isomer thereof to synthesize the compound of Chemical Formula 5-1 and can readily separate from the compound of Chemical Formula 5-1 in a subsequent reaction. The sulfonyl compound available for this reaction may be sulfonyl halides such as sulfonyl chloride, sulfonyl bromide, and sulfonyl iodide, or sulfonyl imides. For example, the sulfonyl compound may include methylsulfonyl halides, ethylsulfonyl halides, propylsulfonyl halides, toluenesulfonyl halides, trifluoromethylsulfonyl halides, trifluoromethylsulfonyl halides, trifluorobenzenesulfonyl halides, N-phenyl bistrifluoromethyl sulfonyl imide, and N-butyl bistrifluoromethyl sulfonyl.

Upon the sulfonation of the compound of Chemical Formula 4-1 or the optical isomer thereof, the sulfonyl compound substitutes for —H of the hydroxyl group (—OH) to give the compound of Chemical Formula 5-1 or the optical isomer thereof. Examples of the sulfonyl group available for the sulfonation may include methylsulfonyl, ethylsulfonyl, propylsulfonyl, toluenesulfonyl, trifluoromethyl sulfonyl, trifluoromethylsulfonyl, ortrifluorobenzene sulfonyl. The sulfonyl group may be preferably methylsulfonyl, p-toluenesulfonyl or trifluoromethylsulfonyl.

The compound of Chemical Formula 4-1 or the optical isomer thereof may react with the sulfonyl compound in the presence of the base. For example, the base may include triethylamine, N,N-diisopropylethylamine, N-methylorphyrin, N-methylpiperidine, 4-dimethylaminopyridine, N,N-dimethylaniline, 2,6-rutidine, tricalcium phosphate, potassium carbonate, pyridine or a mixture thereof. The base may be preferably triethylamine.

Subsequently, the compound of Chemical Formula 5-1 or the optical isomer thereof is oxidized to prepare a compound represented by Chemical Formula 5-2. This oxidation may be carried out by reacting the compound of Chemical Formula 5-1 or the optical isomer thereof with an oxidizing agent. The oxidizing agent available for this oxidation is not limited to specific kinds. Examples of the oxidizing agent may include hydrogen peroxide, metachlorobenzoic acid, manganese compounds such as potassium permanganate, manganese acetate, etc., and a mixture thereof. The oxidation agent may be preferably manganese acetate.

Thereafter, the compound of Chemical Formula 5-2 is reacted with the compound of Chemical Formula 6-1 or the optical isomer thereof in the presence of a base to afford the compound of Chemical Formula 2, the optical isomer thereof, the pharmaceutically acceptable salt thereof, or the hydrate or solvate thereof. The compound of Chemical Formula 5-2 and the compound of Chemical Formula 6-1 may be reacted in an Ullmann coupling manner.

As described above, the optical structure of the compound of Chemical Formula 6-1 determines that of the compound of Chemical Formula 2. Hence, according to the optical structure of the chemical formula 2, the optical structure of the compound of Chemical Formula 6-1 may be adjusted. For example, if the compound of Chemical Formula 2 is intended to be obtained as an (S) enantiomer, the compound of Chemical Formula 6-1 may have an (S)-configuration.

No particular limitations are placed on the kind of the base available for the reaction between the compounds of Chemical Formula 5-2 and 6-1. Examples of the base may include triethylamine, N,N-diisopropylethylamine, N-methylorphyrin, N-methylpiperidine, 4-dimethylaminopyridine, N,N-dimethylaniline, 2,6-rutidine, tricalcium phosphate, potassium carbonate, pyridine, and a mixture thereof. The base may be preferably tricalcium phosphate.

In the case where the compound of Chemical Formula 1 is specified into the compound of Chemical Formula 3, the preparation method of the present invention may be carried out by reacting a compound represented by the following Chemical Formula 4-1 or an optical isomer thereof with a sulfonyl compound to synthesize a compound represented by the following Chemical Formula 5-1 or an optical isomer thereof; and reacting the compound of Chemical Formula 5-1 with a compound represented by the Chemical Formula 6-2 or an optical isomer thereof:

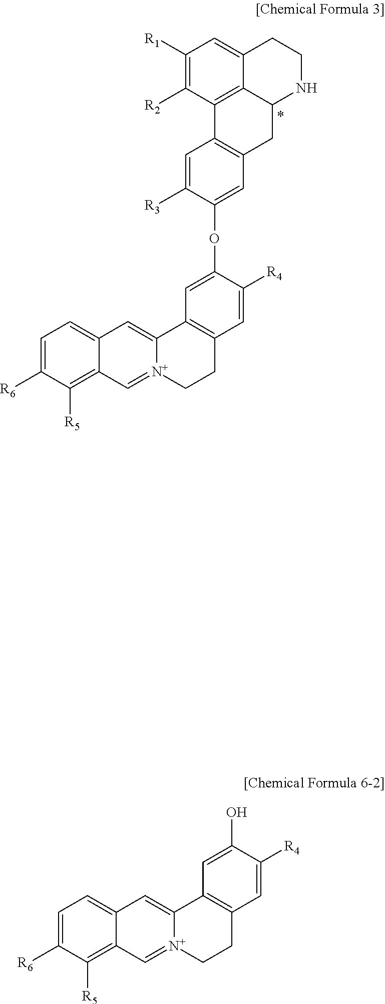

wherein for Chemical Formula 3 or 6-2:

R₁ to R₆ are independently H or methoxy; and

* represents an asymmetric carbon atom.

In accordance with the present invention when the compound of Chemical Formula 1 is specified into the compound of Chemical Formula 3, the preparation method of the present invention may be illustrated in the following Reaction Scheme 2:

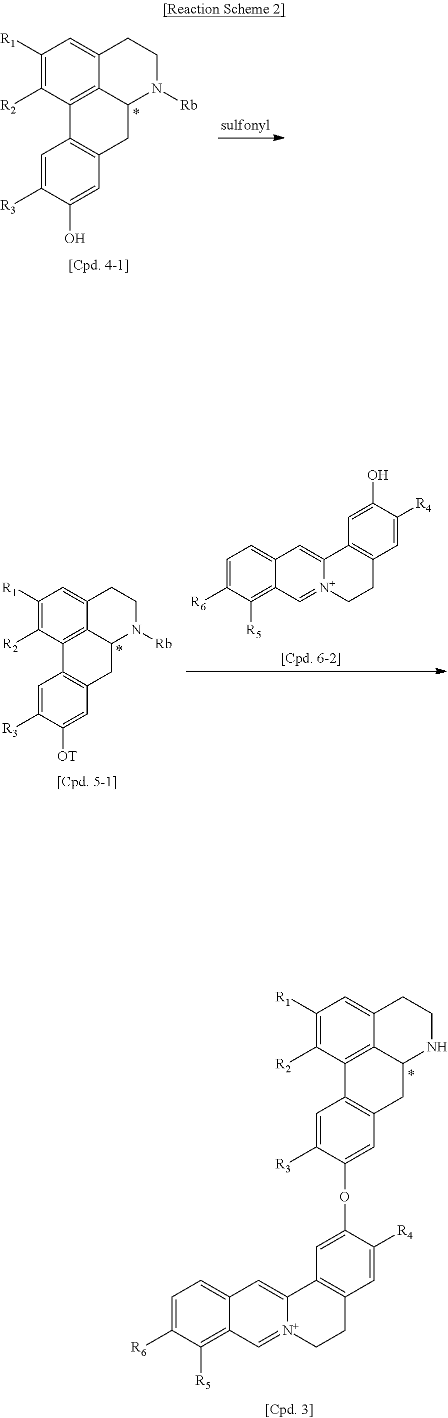

wherein for the reaction scheme 2:

R₁ to R₆ are independently —H or methoxy;

Rb is —H or linear or branched alkyl of C₁ to C₄;

T is linear or branched alkyl sulfonyl of C₁ to C₄, linear or branched fluoroalkyl sulfonyl of C₁ to C₄, or substituted or unsubstituted arylsulfonyl of C₃ to C₁₀; and

* represents an asymmetric carbon atom.

Preferably, R₁ to R₆ are methoxy, Rb is —H, and T is trifluoromethyl sulfonyl in the reaction scheme 2.

In Reaction Scheme 2 according to the preparation method of the present invention, compounds of Chemical Formulas 3, 4-1, and 5-1 bear an asymmetric carbon atom at the position *. Therefore, in accordance with preparing the compound represented by Chemical Formula 3, the optical isomer thereof, the pharmaceutically acceptable salt thereof, or the hydrate or solvate thereof, the compounds represented by Chemical Formula 4-1 and 5-1, or optical isomers thereof may be employed.

The compound of Chemical Formula 4-1, 6-2, or the optical isomers thereof may be prepared using a typical method known in the art, or may be commercially available. For example, the compounds may be obtained from an extract of Corydalis tuber using a well-known method (Tae Ho Lee et al., Biol. Pharm. Bull. 33(6) 958-962 (2010), “Effects of Corydaline from Corydalis Tuber on Gastric Motor Function in an Animal Model”, Ki Hyun Kim et al., Planta Med. 2010 May 27, “New Cytotoxic Tetrahydroprotoberberine-Aporphine Dimeric and Aporphine Alkaloids from Corydalis Turtschaminovii”).

In the preparation method of the present invention, as illustrated in Reaction Scheme 2, the compound of Chemical Formula 4-1 or the optical isomer thereof is reacted with the sulfonyl compound in the presence of a base to give the compound of Chemical Formula 5-1 or the optical isomer thereof. The sulfonyl compound, the sulfonyl group bounded in the compound of Chemical formula 5-1 or the optical compound thereof, and the base used in reaction Scheme 2 may be substantially the same as those which are defined in Reaction Scheme 1.

As can be understood from Reaction Scheme 2 according to the method of present invention, the optical structure of the compound of Chemical Formula 4-1 determines that of the compound represented by Chemical Formula 3. Hence, when the compound of Chemical Formula 4-1 is pure optical isomer, the compound of Chemical Formula 3 is obtained in the form of the predetermined optical structure. For example, if the compound of Chemical Formula 4-1 has an (S)-configuration, the compound of Chemical Formula 3 may be obtained as an (S) enantiomer.

On the other hand, when the compound of Chemical Formula 4-1 is a racemate, the compound of Chemical Formula 3 may be obtained in a racemic mixture. In this case, the racemic mixture can be separated into desired enantiomers of Chemical Formula 3 using a well-known method.

The compound of Chemical Formula 5-1 or the optical isomer is reacted with the compound of Chemical Formula 6-2 or the optical isomer thereof in the presence of the base to prepared the compound of Chemical Formula 3, the optical isomer thereof, the pharmaceutically acceptable salt thereof, or the hydrate or solvate thereof. The compound of Chemical Formula 5-1 or the optical isomer and the compound of Chemical Formula 6-2 may be reacted in an Ullmann coupling manner.

No particular limitations are placed on the kind of the base available for the reaction between the compounds of Chemical Formula 5-1 or the optical isomer and the compounds of Chemical Formula 6-2 of the optical isomer. Examples of the base may include triethylamine, N,N-diisopropylethylamine, N-methylorphyrin, N-methylpiperidine, 4-dimethylaminopyridine, N,N-dimethylaniline, 2,6-rutidine, tricalcium phosphate, potassium carbonate, pyridine, and a mixture thereof. The base may preferably include tricalcium phosphate.

The present invention provides a pharmaceutical composition for the prevention or treatment of a gastrointestinal motility disorder, comprising a novel quinoline derivative compound represented by the following Chemical Formula 1, an optical isomer thereof, a pharmaceutically acceptable salt thereof, or a hydrate or solvate thereof:

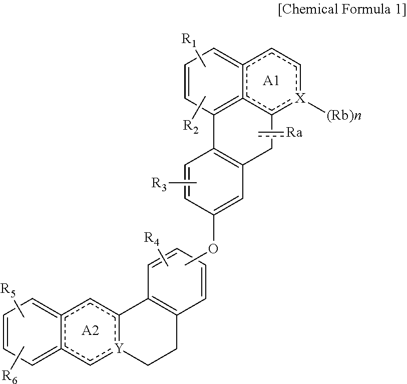

wherein for Chemical Formula 1:

R₁ to R₆ are independently —H or methoxy;

Rₐ is —H, methoxy or —O—;

[symbol] represents a single bond or a double bond, with the proviso that when [symbol] represents the single bond, Ra is —H or methoxy; or [symbol] when represents the double bond, Ra is —O—;

X and Y are independently —N— or —N⁺—;

Rb is —H or linear or branched alkyl of C₁ to C₄;

n means the number of Rb bound with X and is an integer of 0 or 1; and

A1 and A2 are independently a 6-membered cycloaliphatic ring or a 6-membered aromatic ring, with the proviso that when A1 is the 6-membered cycloaliphatic ring, n is 1, or when A1 is the 6-membered aromatic ring, n is zero.

In accordance with the present invention, when A1 or A2 is the cycloaliphatic ring, the quinoline derivative compound of Chemical Formula 1 may bear an asymmetric carbon atom in the cycloaliphatic moiety. In this context, the pharmaceutical composition of the present invention may include the compound represented by Chemical Formula 1, the optical isomer thereof, or the racemate thereof.

In accordance with the present invention, the compound represented by Chemical Formula 1 may be a compound represented by the following Chemical Formula 2 or 3:

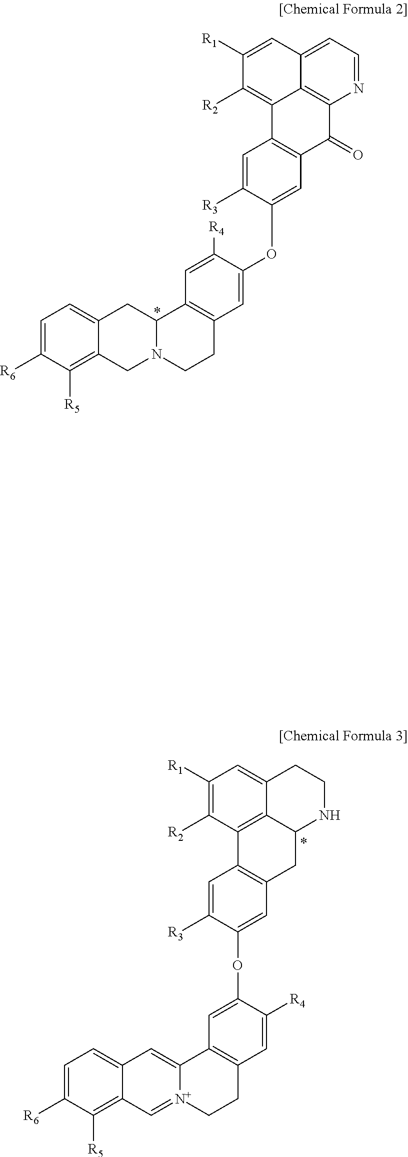

wherein for Chemical Formula 2 or 3:

R₁ to R₆ are independently —H or methoxy; and

* represents an asymmetric carbon atom.

The compound represented by Chemical Formula 2 or 3 may bear an asymmetric carbon atom. The pharmaceutical composition of the present invention may include the compound of Chemical Formula 2 or 3, and its optical isomers.

The pharmaceutical composition of the present invention may include the following compounds, an optical isomer thereof, a pharmaceutically acceptable salt thereof, a hydrate thereof, or a solvate thereof:
1,2,10-Trimethoxy-9-(2,9,10-trimethoxy-5,8,13,13a-tetrahydro-6H-isoquino[3,2-a]isoquinolin-3-yloxy)-dibenzo[de,g]quinolin-7-one; or
3,9,10-Trimethoxy-2-(1,2,10-trimethoxy-5,6,6a,7-tetrahydro-4H-dibenzo[de,g]quinolin-9-yloxy)-5,6-dihydro-isoquino[3,2-a]isoquinolinylium.

The pharmaceutical composition of the present invention may preferably comprise the following compounds, an optical isomer thereof, a pharmaceutically acceptable salt thereof, a hydrate thereof, or a solvate thereof:
(S)-1,2,10-Trimethoxy-9-(2,9,10-trimethoxy-5,8,13,13a-tetrahydro-6H-isoquino[3,2-a]isoquinolin-3-yloxy)-dibenzo[de,g]quinolin-7-one; or
(S)-3,9,10-Trimethoxy-2-(1,2,10-trimethoxy-5,6,6a,7-tetrahydro-4H-dibenzo[de,g]quinolin-9-yloxy)-5,6-dihydro-isoquino[3,2-a]isoquinolinylium.

In the pharmaceutical composition, the pharmaceutically acceptable salt, the hydrate, and the solvate may be as defined above.

The pharmaceutical composition of the present invention may be applicable to the prevention or treatment of functional gastrointestinal disorders, such as gastrointestinal motility disorder.

Examples of gastrointestinal motility disorder may include functional dyspepsia, resulting in symptoms such as early satiety, pain, epigastric distress, a false sense of satiety, heartburn, nausea and vomiting, etc., ulcerative dyspepsia, non-ulcerative dyspepsia, a gastroesophageal reflux disease, reflux esophagitis, gastric atony, intestinal pseudo-obstruction, gastroparesis, constipation; irritable bowel syndrome, general hypersensitive colitis, hypersensitive colitis accompanied by constipation, hypersensitive colitis accompanied by diarrhea, a diabetic gastrointestinal motility disorder, a chemotherapy-induced gastrointestinal motility disorder, a visceral obstruction due to gastrointestinal dysmotility, a gastrointestinal motility disorder due to myotonic dystrophy, and noncardiac chest pain.

Of the gastrointestinal motility disorders, functional dyspepsia does not result in a pathological or biochemical organic lesion, but rather it gives rise to functional symptoms with manifestation of continuous epigastric distress or pain. Medically, this means various symptoms associated with continuous and repetitive discomfort or pain confined to the epigastric part. Specifically, functional dyspepsia may include all the symptoms relating to the digestive system including postprandial fullness, anorexia, abdominal distention, early satiety, belching, epigastric distress or pain, brash, nausea, vomiting, gastric reflux and heartburn, etc.

It is known that about 30% of patients with functional dyspepsia exhibit gastrointestinal dysmotility such as delaying of gastric emptying time taken to transfer foods to the small intestine through the pylorus of the stomach. In addition, early satiety takes place even with a small amount of food intake, due to a decline of gastric accommodation which maintains intragastric pressure by appropriate expansion of the stomach in response to intake of food.

The pharmaceutical composition of the present invention can improve gastric emptying of foods, gastric accommodation, and gastrointestinal transit and activate gastric motility. Therefore, the pharmaceutical composition of the present invention may be effectively prophylactic or therapeutic for functional dyspepsia or various symptoms of gastrointestinal motility disorders.

Moreover, the pharmaceutical composition of the present invention may be capable of improving gastric emptying capacity, gastric accommodation and gastrointestinal transit, without causing adverse side effects such as adverse effects on the heart, thereby contributing to an improvement in gastrointestinal motility.

The pharmaceutical composition of the present invention has high affinity for dopamine-D2 receptors.

Dopamine-D2 receptors, found in the wall of digestive ducts of various mammals, are known to inhibit gastrointestinal motility. Domperidone and metocloprimide, both selective antagonists for the inhibitory receptor dopamine-D2, promote gastrointestinal motility. Also, as dopamine-D2 receptor antagonists that inhibit vomiting, levosulpiride, clebopride, bromopride, etc. are used as antiemetics with prompting gastroprokinetic activity in some countries (P. Moayyedi, S. Soo, J. Deeks, B. Delaney, M. Innes and D. Forman, Pharmacological interventions for non-ulcer dyspepsia, Cochrane Database SystRev 18, 2006; G. Karamanolis and J. Tack, Promotility medications-now and in the future, DigDis 24, 2006 pp. 297-307). Thus, the pharmaceutical composition of the present invention acts as a dopamine-D2 receptor antagonist with high affinity for the dopamine-D2 receptor, so that it can promote gastrointestinal motility.

The pharmaceutical composition of the present invention has high affinity for the serotonin-1B receptor. Sumatriptan, a serotonin-1B receptor agonist, exerts a relaxation effect on the stomach and reduces meal-induced satiety in functional dyspepsia patients, thereby exhibiting a therapeutic activity for impaired gastric accommodation (Tack, H. Piessevaux, B. Coulie, P. Caenepeel and J. Janssens, Role of impaired gastric accommodation to a meal in functional dyspepsia, Gastroenterology 115 (1998), pp. 1346-1352; J. Tack, P. VandenBerghe, B. Coulie and J. Janssens, Sumatriptan is an agonist at 5-HT receptors on myentericneurones in the guinea-pig gastricantrum, Neurogastroenterol Motil (2007), pp. 39-46). Therefore, the pharmaceutical composition of the present invention can be used as a therapeutic or prophylactic agent for impaired gastric accommodation because it exhibits stomach relaxation and a reduction in meal-induced satiety.

The pharmaceutical composition of the present invention may include at least one of the compounds represented by Chemical Formula 1, 2 or 3, the above-suggested compound, optical isomers thereof, pharmaceutically acceptable salts thereof, hydrates thereof, and solvates thereof may be used alone or in combination. For example, the pharmaceutical composition of the present invention one or two ingredients of the compounds represented by Chemical Formula 1, 2 or 3, the above-suggested compound, optical isomers thereof, pharmaceutically acceptable salts thereof, hydrates thereof, and solvates thereof.

In addition to the compounds represented by Chemical Formula 1, 2 or 3, the above-suggested compound, optical isomers thereof, pharmaceutically acceptable salts thereof, hydrates thereof, and solvates, the pharmaceutical composition of the present invention may include another ingredient effective for the prevention or treatment of gastrointestinal motility disorders. For example, the pharmaceutical composition may further comprise domperidone, metoclopramide, levosulpiride, mosapride, itopride, or erythromycin.

The pharmaceutical composition of the present invention may further a pharmaceutically active ingredient effective for the therapy of diseases other than dyspepsia or gastrointestinal motility disorders.

To effect the prevention or treatment of gastrointestinal motility disorders, the pharmaceutical composition of the present invention may be used independently of or in conjunction with a surgical operation, hormonal therapy, a drug regimen, or an agent for biological modulation.

For proper administration, the pharmaceutical composition of the present invention may include at least one pharmaceutically acceptable carrier in addition to the compound of Chemical Formula 1, 2 or 3, the above-suggested compounds, an optical isomer, a pharmaceutically acceptable salt, or a hydrate or solvate thereof. The pharmaceutically acceptable carrier may include saline, sterile water, Ringer's solution, buffered saline, dextrose solution, maltodextrin solution, glycerol, ethanol, or a combination thereof. If necessary, the pharmaceutical composition may further include a typical additive such as an antioxidant, buffer, a bacteriostat, etc.

According to purpose, the pharmaceutical composition of the present invention may be administered orally or parenterally (e.g., intravenously, subcutaneously, intradermally, or topically). The therapeutically effective amount of the active ingredient may vary depending on various factors including a patient's age, weight, gender, health condition, and diet, the time of administration, the route of administration, a period or an interval of the administration, the rate of excretion, constitutional disposition, the property of formulation to be administered, and the severity of disease, etc. The compound of Chemical Formula 1, 2 or 3, an optical isomer thereof, a pharmaceutically acceptable salt thereof, or a hydrate or solvate thereof may be administered in an amount of from 0.01 to 100 mg/kg, and preferably in an amount of from 0.1 to 30 mg/kg once a day, or may be divided till triple doses a day.

For administration, the pharmaceutical composition of the present invention may be formulated into various dosage form. The pharmaceutical composition may be formulated together with a pharmaceutically acceptable carrier into various pharmaceutical dosage forms. The carrier may be a non-toxic, inert formulation auxiliary agent that may be in any phase such as a solid, quasi-solid, or liquid phase, and pharmaceutically acceptable. For example, a filler, a thickener, a binder, a wetting agent, a disintegrant, a dispersant, a surfactant, or a diluent may be employed.

The pharmaceutical composition of the present invention may be prepared into unit dosage forms. In the unit dosage forms, the compound of Chemical Formula 1, 2 or 3, an optical isomer, a pharmaceutically acceptable salt, or a hydrate or a solvate thereof may be present in an amount corresponding to a fraction or a multiple of the daily dose thereof. For example, the unit dosage form may contain the active ingredient in an amount 1, 2, 3 or 4, or 1/2, 1/3 or 1/4 times as much as a necessary daily dose thereof. Preferably, the amount of the active ingredient in a unit dosage form is a single dose which typically corresponds to daily dose or, 1/2, 1/3 or 1/4 of the daily dose.

The pharmaceutical composition of the present invention may be formulated into a tablet, a coated tablet, a capsule, a pill, a granule, a suppository, a liquid, a suspension, an emulsion, a paste, an ointment, a gel, a cream, a lotion, a powder, or a spray. For oral administration, for example, the pharmaceutical composition of the present invention may be formulated into a solid agent, such as a tablet, a pill, a powder, a granule or a capsule, or a liquid agent such a suspension, an internal use liquid, an emulsion or a syrup. Alternatively, the pharmaceutical composition of the present invention may be administered via a parenteral route. For this, the pharmaceutical composition of the present invention may be in the form of an injection, a suspension, an emulsion, a lyophilizate, or a suppository. For example, the compound of Chemical Formula 1, or an optical isomer thereof, a pharmaceutically acceptable salt thereof, a hydrate or a solvate thereof may be formulated together with at least one excipient into a microcapsule.

When the pharmaceutical composition of the present invention is formulated into a solid preparation such as a tablet, a coated tablet, a capsule, a pill or a granule, the active ingredient may be combined with (a) a filler and a thickener such as starch, lactose, sucrose, glucose, mannitol, or silicate, (b) a binder, such as carboxymethyl cellulose, alginate, gelatin, polyethylene glycol, microcrystalline cellulose, highly dispersible silica, natural gum, synthetic gum, povidone, copovidone, polyvinyl pyrrolidone, or gelatin, (c) a moisture absorbent such as glycerol, (d) a disintegrant such as agar, calcium carbonate, or sodium carbonate, (e) a dissolution retardant such as paraffin, (f) an absorption accelerator such as a quaternary ammonium compound, (g) a wetting agent such as cetyl alcohol, or glycerol monostearate, (h) an absorber such as kaolin or bentonite, or (i) a lubricant such as talc, calcium stearate, magnesium stearate, or a solid polyethylene glycol, or a mixture of (a) to (i).

For formulating the pharmaceutical composition of the present invention into a liquid agent for oral administration, such as a suspension, an internal use liquid, or a syrup, various additives including a diluent such as water, liquid paraffin, etc., a wetting agent, a sweetener, an odorant, a preservative, an antiseptic and a colorant may be added as necessary. For example, peppermint oil, eucalyptus oil or a sweetener such as saccharin may be added to the pharmaceutical composition of the present invention.

When the pharmaceutical composition of the present invention is formulated into a suppository, a water-soluble or insoluble excipient, for instance, a lipid such as polyethylene glycol, cacao lipid, etc., a high ester (e.g., C₁₄-alcohol with C₁₆-fatty acid), Witepsol, macrogol, Tween 61, laurin butter, glycerol gelatin, or a combination thereof may be employed.

Also, the pharmaceutical composition of the present invention may take the form of ointments, pastes, creams or gels. In this regard, animal or vegetable lipids, wax paraffins, starch, tragacanth, cellulose derivatives, polyethylene glycol, silicon, bentonite, silicic acid, talc, zinc oxide or a combination thereof may be used.

For a powder or spray, the pharmaceutical composition of the present invention may be formulated in combination with lactose, talc, silicic acid, aluminum oxide, calcium silicate, polyamide powder, or a mixture thereof. A spray formulation may further comprise a typical propellant such as chlorofluorohydrocarbon. PEG-4000 and glycerin may be typically needed to obtain an inhalation spray.

In the case of liquid or emulsion formulations intended for the parenteral administration of the pharmaceutical composition of the present invention, their formulations may be obtained with a solvent, a dissolving agent, or an emulsifier. For example, water, ethyl alcohol, isopropyl alcohol, ethyl carbonate, ethyl acetate, benzyl alcohol, benzyl benzoate, propylene glycol, 1,3-butylene glycol, dimethyl formamide, an oil such as cotton seed oil, peanut oil, corn seed oil, olive oil, castor oil, or sesame oil, glycerol, glycerol formalcohol, tetrahydrofurfuryl alcohol, polyethylene glycol, a fatty acid of sorbitan, or a combination thereof may be used to formulate the composition of the present invention into a liquid or an emulsion.

The liquid or emulsion for parenteral administration may be in a sterile, and blood-isotonic state.

For use in formulating the pharmaceutical composition into a suspension, a liquid diluent, such as water, ethyl alcohol, propylene glycol, or polyethylene glycol, ethoxylated isostearyl alcohol, polyoxyethylene sorbitol, and sorbitan ester, microcrystalline cellulose, aluminum metahydroxide, bentonite, agar, tragacanth, an injectable ester such as ethyl oleate, and a mixture thereof may be suitable.

In addition, the pharmaceutical composition of the present invention may be formulated into a sustained or immediate release form with an excipient, a diluent, a dispersant, a surfactant, a binder, a lubricant or an additive. For example, the pharmaceutical of the present invention may be formulated into the sustained or immediate release form in order to release the compound of Chemical Formula 1, 2 or 3, an optical isomer, a pharmaceutically acceptable salt, or a hydrate or a solvate thereof in the predetermined organ of the body.

The pharmaceutical composition of the present invention may be formulated into the sustained release form using a sustained release agent such as an enteric coating agent, a water-insoluble polymer, a hydrophobic compound, a polymeric material such as a hydrophilic polymer, or an embedding agent such as wax. For example, the pharmaceutical composition takes a form of tablets, capsules, pills or granules, a coating film may be applied to them to achieve a sustained release.

No particular limitations are imparted to the amount of the additives used in preparing a formulation of the pharmaceutical composition of the present invention, such as carriers, fillers, thickeners, binders, wetting agents, disintegrants, dispersants, surfactants, or diluents. The amount of additives may be suitably adjusted within the range typically used in typical formulations.

Using a suitable method known in the art, the pharmaceutical composition of the present invention may be formulated depending on the disease to be treated or the ingredient to be used. For example, the compound of Chemical Formula 1, 2 or 3, the above-suggested compound, or an optical isomer or a pharmaceutically acceptable salt thereof may be mixed with an excipient to be formulated into a desired preparation.

In the pharmaceutical composition of the present invention, the active ingredient, such as the compound of Chemical Formulas 1, 2 or 3, the above-suggested compound, an optical isomer, a pharmaceutically acceptable salt, or a hydrate or a solvate thereof, may be employed in an amount of from about 0.1 to about 99.5 wt %, and preferably in an amount of from about 0.5 wt % to about 95 wt %.

Also, the present invention provides the use of the pharmaceutical composition including the compound of Chemical Formula 1, 2 or 3, the above-suggested compound, an optical isomer, a pharmaceutically acceptable salt, or a hydrate or a solvate thereof in the treatment of a gastrointestinal motility disorder.

The present invention provides a method for preventing or treating dyspepsia or a gastrointestinal motility disorder, using the compound of Chemical Formula 1, 2 or 3, the above-suggested compound, an optical isomer, a pharmaceutically acceptable salt, a hydrate or a solvate thereof. The pharmaceutical composition may be administered into the subject including a human to prevent or treat dyspepsia or a gastrointestinal motility disorder.

### Advantageous Effects

As described hitherto, the novel quinoline derivative compounds, optical isomers thereof, pharmaceutically acceptable salts thereof, and hydrates or solvates thereof can activate gastrointestinal motility. Compositions comprising these gastroprokinetic agents can be effectively applied to the prophylaxis and therapy of functional dyspepsia or various symptoms of gastrointestinal motility disorders.

### MODE FOR INVENTION

A better understanding of the present invention may be obtained through the following examples which are set forth to illustrate, but are not to be construed as limiting the present invention.

Unless stated otherwise, reagents and solvents used in the following Example section were purchased from Sigma-Aldrich. (+)-N-methyllaurotetanine, (+)-laurotetanine, (−)-corypalmine, and columbamine were obtained from an extract of Corydalis tuber using a well-known method (Tae Ho Lee et al., Biol. Pharm. Bull. 33(6) 958-962 (2010), “Effects of Corydaline from Corydalis Tuber on Gastric Motor Function in an Animal Model,” Ki Hyun Kim et al., Planta Med. 2010 May 27, “New Cytotoxic Tetrahydroprotoberberine-Aporphine Dimeric and Aporphine Alkaloides from Corydalis Turtschaminovii”).

¹H-NMR data were measured using a UNITY INOVA 500 NMR spectrometer, Varian. IR data measured using an IFS-66/S FT-IR spectrometer, Bruker, UV data measured using UV-1601 UV-visible spectrophotometer, Shimadzu, and CD data measured using a J-715 spectropolarimeter, JASCO. For mass data, a JMS700 mass spectrometer, JEOL, was employed.

### Example 1

### Preparation of (S)-1,2,10-Trimethoxy-9-(2,9,10-trimethoxy-5,8,13,13a-tetrahydro-6H-isoquino[3,2-a]isoquinolin-3-yloxy)-dibenzo[de,g]quinolin-7-one)

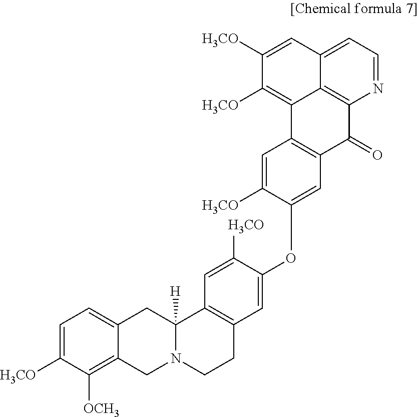 [SMILES: COc1cc2c(cc1Oc1cc3c(cc1OC)-c1c(OC)c(OC)cc4ccnc(c14)C3=O)CCN1Cc3c(ccc(OC)c3OC)C[C@@H]21]

### Step 1: Synthesis of (S)-1,2,10-trimethoxy-6-methyl-5,6,6a,7-tetrahydro-4H-dibenzo[de,g]quinolin-9-yl-trifluoro-methanesulfonate

In 10 ml of dichloromethane was dissolved 200 mg of (S)-1,2,10-trimethoxy-6-methyl-5,6,6a,7-tetrahydro-4H-dibenzo[de,g]quinolin-9-ol((+)-N-methyllaurotetanine), followed by adding 194 mg of potassium carbonate, 0.2 ml of triethylamine and 251 mg of N-phenyl-bis trifluoro-methylsulfone imide at 0° C. and then by stirring the mixture at room temperature for 3 days. After completion of the reaction, the reaction mixture was extracted with water and dichloromethane. The organic layer was washed with saturated brine, and dried over anhydride magnesium sulfate. Thereafter, the solvent was removed by distillation under reduced pressure. Then 194 mg of the title compound was obtained by silica gel column chromatography.

### Step 2: Synthesis of 1,2,10-trimethoxy-7-oxo-dibenzo[de,g]quinolin-9-yl trifluoromethanesulfonate

In 10 ml of acetic acid was dissolved 194 mg of (S)-1,2,10-trimethoxy-6-methyl-5,6,6a,7-tetrahydro-4H-dibenzo[de,g]quinolin-9-yl-trifluoromethanesulfonate synthesized in Step 1, followed by adding 880 mg of manganese acetate and then by stirring at 70° C. for 2 hrs. After completion of the reaction, acetic acid was removed by distillation under a reduced pressure. The residue was diluted in chloroform and extracted with a saturated sodium hydrogen carbonate solution. The organic layer thus formed was washed with water, and dried over anhydride magnesium sulfate. The solvent was removed by distillation under a reduced pressure. Then 143 mg of the title compound was obtained by silica gel column chromatography

### Step 3: Synthesis of (S)-1,2,10-trimethoxy-9-(2,9,10-trimethoxy-5,8,13,13a-tetrahydro-6H-isoquino[3,2-a]-isoquinolin-3-yloxy)-dibenzo[de,g]quinolin-7-one

In 10 ml of toluene were dissolved 3.5 mg of palladium acetate and 4.6 mg of 2-(di-tert-butylphosphino)biphenyl, followed by adding 143 mg of 1,2,10-trimethoxy-7-oxo-dibenzo[de, g]quinolin-9-yl-trifluoromethanesulfonate synthesized in Step 2, 116 mg of (−)-corypalmine and 130 mg of tripotassium phosphate and then stirring at 95° C. for 18 hrs. The reaction mixture was diluted in diethylether and filtered. The filtrate was concentrated under reduced pressure. Then 122 mg of the compound represented by Chemical Formula 7 was obtained by silica gel column chromatography.

IR (KBr): vₘₐₓ=3364, 2945, 1648, 1510, 1460, 1279, 1056, 675 cm-1

UV λₘₐₓ (MeOH) nm (log ε): 204 (4.17), 236 (3.52), 277 (4.11), 311 (4.21), 363 (4.38);

CD (EtOH) λmax (Δε): 204 (−29.8), 235 (−4.0), 280 (+1.9) nm;

FAB-MS: m/z=661[M+H]⁺;

HR-FAB-MS: m/z=661.2557[M+H]⁺ (calcd. for C₃₉H₃₇N₂O₈: 661.2550);

¹H-NMR (CD₃OD, 500 MHz): 2.56 (td, 1H), 2.66 (m, 1H), 2.80 (dd, 1H), 3.11 (m, 1H), 3.19 (m, 1H), 3.24 (dd, 1H), 3.52 (dd, 1H), 3.55 (d, 1H), 3.86 (s, 3H), 3.87 (s, 3H), 3.88 (s, 3H), 4.03 (s, 3H), 4.04 (s, 3H), 4.05 (s, 3H), 4.24 (d, 1H), 6.60 (s, 1H), 6.79 (d, 1H), 6.84 (s, 1H), 6.87 (d, 1H), 7.22 (s, 1H), 7.79 (d, 1H), 8.05 (s, 1H), 8.84 (s, 1H), 8.92 (d, 1H);

¹³C-NMR (CD₃OD, 125 MHz): 29.1, 36.2, 51.6, 54.0, 55.9, 56.2, 56.3, 56.7, 59.2, 60.1, 60.6, 106.0, 109.9, 110.2, 110.6, 110.9, 111.3, 120.1, 121.7, 123.3, 123.9, 126.0, 127.0, 127.1, 128.2, 129.0, 129.1, 135.4, 143.9, 145.0, 145.0, 145.6, 149.3, 149.6, 150.2, 150.9, 153.8, 156.7, 181.4.

### Example 2

### Preparation of (S)-3,9,10-Trimethoxy-2-(1,2,10-trimethoxy-5,6,6a,7-tetrahydro-4H-dibenzo[de,g]quinolin-9-yloxy)-5,6-dihydro-isoquino[3,2-a]isoquinolinylium

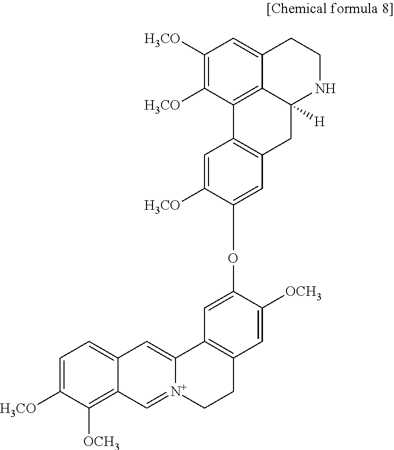 [SMILES: COc1cc2c(cc1Oc1cc3c(cc1OC)CC[n+]1cc4c(OC)c(OC)ccc4cc1-3)C[C@@H]1NCCc3cc(OC)c(OC)c-2c31]

### Step 1: Synthesis of (S)-1,2,10-trimethoxy-5,6,6a,7-tetrahydro-4H-dibenzo[de,g]quinolin-9-yl trifluoromethanesulfonate

In 10 ml of dichloromethane was dissolved 200 mg of (S)-1,2,10-trimethoxy-5,6,6a,7-tetrahydro-4H-dibenzo[de,g]quinolin-9-ol((+)-laurotetanine) followed by adding 203 mg of potassium carbonate, 0.2 ml of triethylamine, and 262 mg of N-phenyl-bis trifluoromethylsulfone imide at 0° C. and then by stirring at room temperature for 24 hrs. After completion of the reaction, the reaction mixture was extracted with water and dichloromethane. The organic layer was washed with saturated brine, and dried over anhydride magnesium sulfate. Thereafter, the solvent was removed by distillation under reduced pressure. Then, 202 mg of the title compound was obtained by silica gel column chromatography.

### Step 2: Synthesis of (S)-3,9,10-trimethoxy-2-(1,2,10-trimethoxy-5,6,6a,7-tetrahydro-4H-dibenzo[de,g]quinolin-9-yloxy)-5,6-dihydro-isoquino[3,2-a]isoquinolium

In 15 ml of toluene were dissolved 4.9 mg of palladium acetate and 6.6 mg of 2-(di-tert-butylphosphino)biphenyl, followed by adding 202 mg of (S)-1,2,10-trimethoxy-5,6,6a,7-tetrahydro-4H-dibenzo[de,g]quinolin-9-yl trifluoromethanesulfonate synthesized in Step 1, 164 mg of columbamine and 187 mg of tripotassium phosphate and then by stirring at 95° C. for 18 hrs. The reaction mixture was diluted in diethylether and filtered. The filtrate was concentrated under reduced pressure. Then 157 mg of the compound represented by Chemical Formula 8 was obtained by silica gel column chromatography.

IR (KBr): vₘₐₓ=3386, 2945, 1647, 1511, 1457, 1276, 1028, 675 cm-1;

UV λₘₐₓ (MeOH) nm (log ε): 205 (4.23), 220 (4.10), 273 (4.23), 297 (4.51), 307 (3.78);

CD (MeOH) λₘₐₓ (Δε) 210 (+12.0), 236 (+18.4), 261 (−8.1), 275 (−16.4), 310 (+25.7) nm;

FAB-MS: m/z=648[M+H]+;

HR-FAB-MS: m/z=648.2841[M+H]+ (calcd. for C₃₉H₄0N₂O₇: 648.2836);

¹H-NMR (CD₃OD, 500 MHz): 2.50 (m, 1H), 2.79 (m, 1H), 2.80 (t, 1H), 3.05 (dd, 1H), 3.26 (m, 1H), 3.29 (t, 2H), 3.62 (s, 3H), 3.66 (m, 1H), 3.86 (s, dd, 4H), 3.91 (s, 3H), 3.96 (s, 3H), 4.12 (s, 3H), 4.24 (s, 3H), 5.25 (t, 2H), 6.77 (s, 2H), 6.81 (s, 1H), 7.51 (s, 1H), 7.73 (d, 1H), 8.01 (d, 1H), 8.08 (s, 1H), 8.74 (s, 1H), 10.34 (s, 1H);

₁₃C-NMR (CD₃OD, 125 MHz): 27.3, 27.3, 35.5, 53.3, 56.0, 56.0, 56.4, 56.2, 57.0, 57.2, 60.6, 62.0, 108.6, 110.1, 111.1, 111.3, 111.8, 119.1, 120.1, 122.2, 122.8, 123.4, 126.2, 127.0, 127.2, 127.9, 129.4, 129.6, 133.7, 137.4, 145.1, 145.2, 146.3, 147.5, 149.0, 150.5, 151.3, 152.3, 152.7

Although the following provides a more detailed explanation of the present invention by indicating Experimental Examples, the present invention is not limited thereto.

### Experimental Example 1

### Assay for Affinity for Dopamine Receptor

Affinity of the compounds of the present invention for the dopamine-D2 receptor, which is found in the wall of the gastric tract of mammals and inhibits gastrointestinal motility, was determined by measuring competitive inhibition against the binding to the receptor of a radio-labeled ligand which is known for its affinity for the receptor. In this regard, the experiment was performed according to the method described in [Heys G et al. Mol Endocrinol. 6:920, 1992] and [Grandy D K et al. Proc Natl Acad Sci USA. 86:9762, 1989].

In tris-HCl buffer (50 nM Tris-HCl, pH 7.4, 120 mM NaCl, 5 mM KCl, 2 mM CaCl₂, 1 mM MgCl₂) 150 μg of Chinese hamster ovary (CHO) cells transfected with a human dopamine D2ₛ gene were incubated at 25° C. for 120 min with 0.16 nM of tritium Spiperone (³[H] Spiperon [NEN, 250 μCi]) and 10 μM of the compound of interest of the present invention. After completion of the reaction, the reaction mixture was filtered through Whatman GF/B filter (unifilter-96, Lot: 6005177, PerkinElmer) to separate ligand-bound receptors. These bound receptors were washed three times with a Tris-HCl buffer. Then 10 mL of a scintillation cocktail (Lot: 03999, Fluka) was added and reacted for 16 hrs or longer to measure radiation activity using a beta-counter (Packcard) in order to calculate the quantity of tritium spiperone bound to the receptor. Meanwhile, 10 μM of Haloperidol (H1512, Sigma) was incubated in the presence of 0.16 nM tritium Spiperone to determine a nonspecific binding value. The experiment was independently carried out twice, and receptor affinity was calculated using the following Equation 1. A mean value of the measurements is given in Table 1, below.

Receptor Affinity(%)={(Total CPM−Nonspecific Binding CPM−CPM of tritium Spiperone remaining after addition of interest compound of the present invention)/(Total CPM−Nonspecific Binding CPM)}×100 Equation 1

(CPM: counter per minute)

TABLE 1

| Example | Affinity (%) |
|---|---|
| Compound of Example 1 | 91 |
| Compound Of Example 2 | 25 |
| Itopride | 75 |

As can be seen in Table 1, the compounds of the present invention at a concentration of 10 μM exhibited high affinity for the dopamine-D2 receptor. Particularly, the compound of Example 1 was observed to have far higher affinity for the dopamine-D2 receptor than does the dopamine-D2 antagonist itopride, conventionally used as a therapeutic for functional dyspepsia. It is understood from the data that the compounds of the present invention function as a gastroprokinetic agent like the conventional dopamine-D2 antagonists, such as domperidone, metochlopramide, or itopride, promoting gastrointestinal motility.

### Experimental Example 2

### Assay for Affinity for Serotonin-1B Receptor

Compounds of the present invention were examined for affinity for the serotonin-1B receptor which can exert a relaxation effect on the stomach to relieve impaired gastric accommodation of functional dyspepsia patients in response to an agonist.

In a Tris-HCl buffer (50 mM Tris-HCl, pH 7.4, 120 mM NaCl, 5 mM KCl, 2 mM CaCl₂, 1 mM MgCl₂), 4 mg of cell membranes of the cerebral cortex excised from male Winstar rats, each weighing 175±25 g, was incubated at 37° C. for 1.5 hrs with 10 pM of ¹²⁵[I] cyanopindolol (PerkinElmer, 100 μCi) and 10 μM of the compound of interest of the present invention per well. After completion of the incubation, the incubation mixture was filtered through Whatman GF/B filter (unifilter-96, Lot: 6005177, PerkinElmer) to separate ligand-bound receptors. These bound receptors were washed three times with 5 mL of a Tris-HCl buffer. Then, 10 mL of a scintillation cocktail (Lot: 03999, Fluka) was added and reacted for 16 hrs or longer to measure radiation activity using a beta-counter (Packcard) in order to calculate the quantity of ¹²⁵[I] cyanopindolol bound to the receptor.

Meanwhile, 10 μM of serotonin (H9523, Sigma) was incubated in the presence of 10 pM of ¹²⁵[I] cyanopindolol to determine a nonspecific binding value. The experiment was independently carried out twice, and receptor affinity was calculated using the following Equation 2. A mean value of the measurements is given in Table 2, below.

Receptor Affinity(%)={(Total CPM−Nonspecific Binding CPM−CPM of ¹²⁵[I] cyanopindolol remaining after addition of Interest compound of the present invention)/(Total CPM−Nonspecific Binding CPM)}×100(CPM: counter per minute) [Equation 2]

TABLE 2

| test compound | Affinity (%) |
|---|---|
| Compound of Example 1 | 61 |
| Compound Of Example 2 | 35 |

As can be seen in Table 2, the compounds of the present invention at a concentration of 10 μM exhibited a high affinity for the serotonin-1B receptor. Thus, it is understood from the data that the compounds of the present invention can bind to the serotonin-1B to exert a relaxation effect on the stomach and thus a palliative effect on impaired gastric accommodation.

### Experimental Example 3

### Gastric Emptying Effect on Normal Rat

Gastric emptying activity of the compounds of the present invention was examined in the following experiment.

Sprague-Dawley rats, each weighing 200˜250 g, were acclimated for one week at a temperature of 22˜24° C. and a humidity of 60-80% while a standard diet and water were provided, and then were divided into groups according to weight. Thereafter, they were starved for 24 hrs before an experiment while water was provided. From 3 hrs before the experiment, no more water was provided. A 3% hydroxy propyl methyl cellulose solution containing the compound prepared in Example 1 or 2 in an amount of 10 μg/kg was orally administered at a dose of 1 mL to each rat. For control 1, only 1 mL of a 3% hydroxypropylmethyl cellulose solution was used while sisapride was orally administered at a dose of 1 mg/kg in 1 mL of a 3% hydroxypropylmethyl cellulose to control 2.

After 45 min, a semi-solid test meal prepared by mashing feeds in water, was orally administered in an amount of 2 mL to each rat. After 35 min, the SD rats were killed, and the stomach containing the semi-solid test meal was excised, and weighed. Subsequently, the stomach was washed with distilled water to remove the gut content, dried, and weighed again, in order to calculate the weight of the semi-solid test meal left in the gut. Gastric emptying rate was calculated according to the following Equation 3, and the results are summarized in Table 3, below.

Gastric Emptying(%)=[1−(weight of semi-solid test meal left in gut/weight of semi-solid test meal at 0-time)]×100 [Equation 3]

(weight of semi-solid test meal at 0-time means weight of the semi-solid test meal left in the rat killed immediately after the administration of the semi-solid test meal)

TABLE 3

| Example | Dose | Gastric Emptying (%) |
|---|---|---|
| Compound of Example 1 | 10 (μg/kg) | 62.3 |
| Compound of Example 2 | 10 (μg/kg) | 47.3 |
| Control 1 | — | 25.4 |
| Control 2 | 10 mg/kg | 68.9 |

As can be seen in Table 3, the compound prepared in Example 1 or 2 increased gastric emptying roughly two-fold, compared to control 1, to which the drug was not administered. Particularly, the compound of Example 1 exhibited a gastric emptying rate similar to that of the conventional therapeutic sisapride even though it was administered at 1/1000 of the dose of sisapride. These data demonstrate that the compounds of Examples 1 and 2 function to promote gastrointestinal motility to release the meal from the stomach.

### INDUSTRIAL APPLICABILITY

As described hitherto, the novel quinoline derivative compounds, optical isomers thereof, pharmaceutically acceptable salt thereof, and hydrates or solvates thereof according to the present invention act as gastroprokinetic agents to activate gastrointestinal motility. Thus, compositions comprising the novel quinoline derivative compounds, optical isomers thereof, pharmaceutically acceptable salt thereof, and hydrates or solvates thereof can be effectively applied to the prophylaxis and therapy of functional dyspepsia or various symptoms of gastrointestinal motility disorders.

1. A quinoline derivative compound represented by the following Chemical Formula 1, an optical isomer thereof, a pharmaceutically acceptable salt thereof, and a hydrate or solvate thereof: 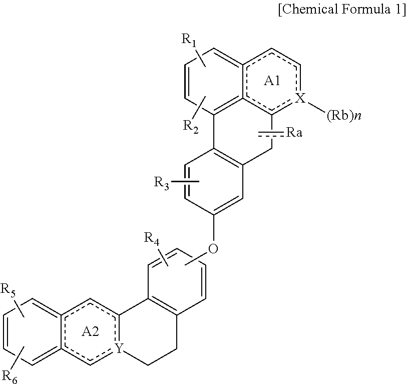
wherein for Chemical Formula 1:
R₁ to R₆ are independently —H or methoxy;
Rₐ is —H, methoxy or —O—; [symbol]
represents a single bond or a double bond, with the proviso that when [symbol] represents the single bond, Ra is —H or methoxy; or when [symbol] represents the double bond, Ra is —O—,
X and Y are independently —N— or —N⁺—;
Rb is —H or linear or branched alkyl of C₁ to C₄;
n means the number of Rb bound with X and is an integer of 0 or 1; and
A1 and A2 are independently a 6-membered cycloaliphatic ring or a 6-membered aromatic ring, with the proviso that when A1 is the 6-membered cycloaliphatic ring, n is 1, or when A1 is the 6-membered aromatic ring, n is zero.

2. The quinoline derivative compound of claim 1 represented by Chemical Formula 1, the optical isomer, the pharmaceutically acceptable salt, or the hydrate or solvate thereof, wherein the quinoline derivative compound represented by Chemical Formula 1 is a compound represented by the following Chemical Formula 2 or 3: 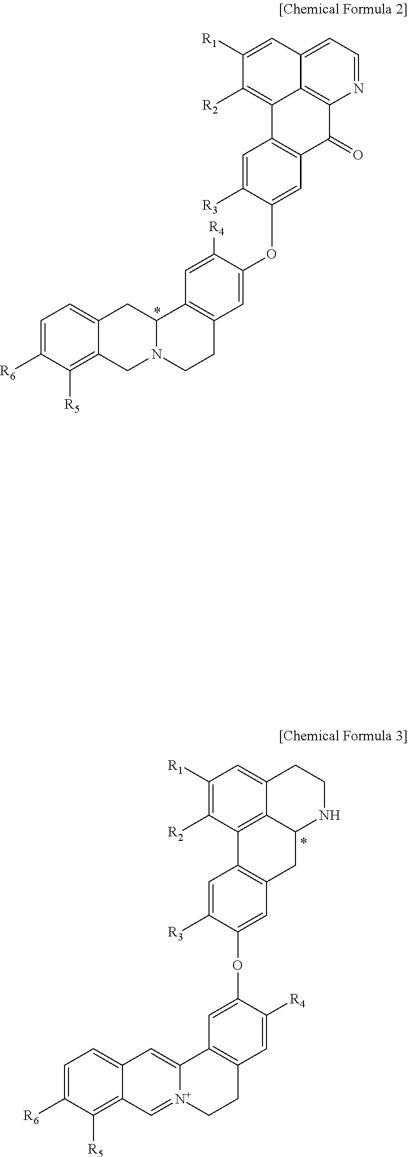
wherein for Chemical Formulas 2 and 3, R₁ to R₆ are independently —H or methoxy.

3. The quinoline derivative compound of claim 1 represented by Chemical Formula 1, the optical isomer, the pharmaceutically acceptable salt, or the hydrate or solvate thereof, wherein the quinoline derivative compound represented by Chemical Formula 1 is (S)-1,2,10-Trimethoxy-9-(2,9,10-trimethoxy-5,8,13,13a-tetrahydro-6H-isoquino[3,2-a]-isoquinolin-3-yloxy)-dibenzo[de,g]quinolin-7-one or (S)-3,9,10-Trimethoxy-2-(1,2,10-trimethoxy-5,6,6a,7-tetrahydro-4H-dibenzo[de,g]quinolin-9-yloxy)-5,6-dihydro-isoquino[3,2-a]-isoquinolinylium.

4. A method for preparing a compound represented by the following Chemical Formula 1, an optical isomer thereof, a pharmaceutically acceptable salt thereof, or a hydrate or solvate thereof, comprising:
1) reacting a compound represented by the following Chemical Formula 4 or an optimal isomer thereof with a sulfonyl compound in presence of a base to synthesis a compound represented by the following Chemical Formula 5 or an optical isomer thereof; and
2) reacting the compound of Chemical Formula 5 or the optimal isomer thereof with a compound represented by the following Chemical Formula 6 in presence of a base: 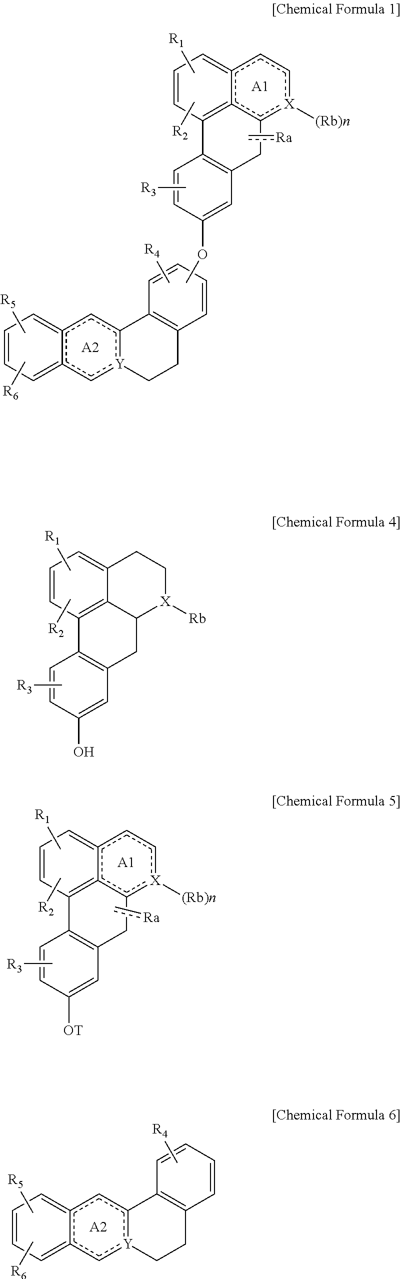
wherein for Chemical Formulas 1, 4, 5 and 6:
R₁ to R₆ are independently H or methoxy;
Ra is —H, methoxy or —O—; [symbol]
represents a single bond or a double bond, with the proviso that when [symbol] represents a single bond, Ra is —H or methoxy; or when [symbol] represents a double bond, Ra is —O—;
X and Y are independently —N— or —N⁺—;
Rb is —H or linear or branched alkyl of C₁ to C₄;
n means the number of Rb bound with X and is an integer of 0 or 1;
A1 and A2 are independently a 6-membered cycloaliphatic ring or a 6-membered aromatic ring, with the proviso that when A1 is the 6-membered cycloaliphatic ring, n is 1, or when A1 is the 6-membered aromatic ring, n is zero; and
T is linear or branched alkyl sulfonyl of C₁ to C₄, linear or branched fluoroalkyl sulfonyl of C₁ to C₄, or substituted or unsubstituted arylsulfonyl of C₃ to C₁₀.

5. The method of claim 4, wherein the step 1) comprises:
reacting the compound represented by Chemical Formula 4 or the optical isomer thereof with the sulfonyl compound to synthesize a compound represented by the following Chemical Formula 5-1 or an optical isomer thereof; and
oxidizing the compound of Chemical Formula 5-1 or the optical isomer thereof to prepare a compound represented by the following Chemical Formula 5-2: 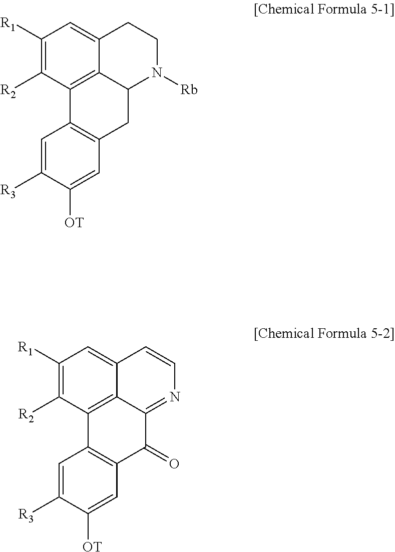
wherein for Chemical Formulas 5-1 and 5-2:
R₁ to R₃ are independently —H or methoxy;
Rb is —H or a linear or branched alkyl of C₁ to C₄; and
T is linear or branched alkyl sulfonyl of C₁ to C₄, linear or branched fluoroalkyl sulfonyl of C₁ to C₄, or substituted or unsubstituted arylsulfonyl of C₃ to C₁₀.

6. The method of claim 5, wherein the step 2) comprises reacting the compound represented by Chemical Formula 5-2 with a compound represented by the following Chemical Formula 6-1 or an optical isomer thereof: 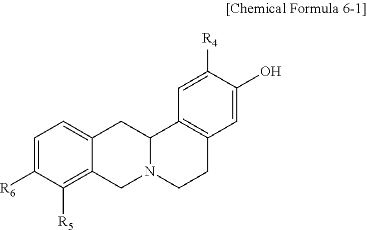
wherein for Chemical Formula 6-1, R₄ to R₆ are independently —H or methoxy.

7. The method of claim 4, wherein the step 1) comprises reacting the compound of Chemical Formula 4 or the optical isomer thereof with the sulfonyl compound to synthesize a compound represented by the following Chemical Formula 5-1 or the optical isomer thereof: 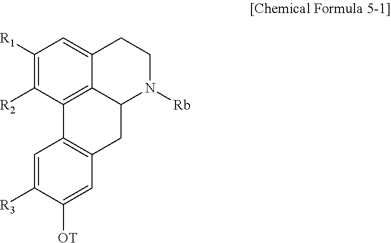
wherein for Chemical Formula 5-1:
R₁ to R₃ are independently —H or methoxy;
Rb is —H or a linear or branched alkyl of C₁ to C₄; and
T is linear or branched alkyl sulfonyl of C₁ to C₄, linear or branched fluoroalkyl sulfonyl of C₁ to C₄, or substituted or unsubstituted arylsulfonyl of C₃ to C₁₀.

8. The method of claim 7, wherein the step 2) comprises reacting the compound of Chemical Formula 5-1 or the optical isomer thereof with a compound represented by the following Chemical Formula 6-2: 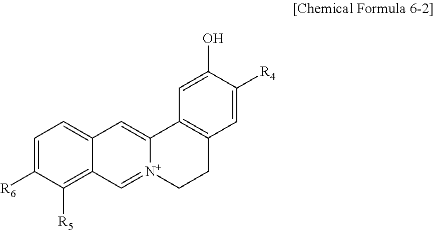
wherein for Chemical formula 6-2, R₄ to R₆ are independently H or methoxy.

9. A pharmaceutical composition for the prevention or treatment of a gastrointestinal motility disorder, comprising a novel quinoline derivative compound represented by the following Chemical Formula 1, an optical isomer thereof, a pharmaceutically acceptable salt thereof, or a hydrate or solvate thereof: 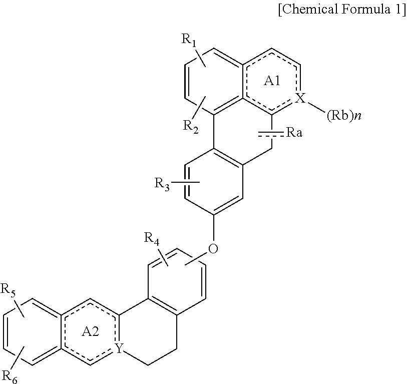
wherein for Chemical Formula 1:
R₁ to R₆ are independently —H or methoxy;
Rₐ is —H methoxy or —O—; [symbol]
represents a single bond or a double bond, with the proviso that when [symbol] represents the single bond, Ra is —H or methoxy; or when [symbol] represents the double bond, Ra is —O—;
X and Y are independently —N— or —N⁺—;
Rb is —H or linear or branched alkyl of C₁ to C₄,
n means the number of Rb bound with X and is an integer of 0 or 1; and
A1 and A2 are independently a 6-membered cycloaliphatic ring or a 6-membered aromatic ring, with the proviso that when A1 is the 6-membered cycloaliphatic ring, n is 1, or when A1 is the 6-membered aromatic ring, n is zero.

10. The pharmaceutical composition of claim 9, wherein the quinoline derivative compound represented by Chemical Formula 1 is (S)-1,2,10-Trimethoxy-9-(2,9,10-trimethoxy-5,8,13,13a-tetrahydro-6H-isoquino[3,2-a]isoquinolin-3-yloxy)-dibenzo[de,g]quinolin-7-one or (S)-3,9,10-Trimethoxy-2-(1,2,10-trimethoxy-5,6,6a,7-tetrahydro-4H-dibenzo[de,g]quinolin-9-yloxy)-5,6-dihydro-isoquino[3,2-a]isoquinolinylium.
